(19) United States
(12) Patent Application Publication
(10) Pub. No.: US 2011/0112325 A1
(43) Pub. Date: May 12, 2011

(54) CATALYST FOR PRODUCING ACROLEIN AND/OR ACRYLIC ACID AND PROCESS FOR PRODUCING ACROLEIN AND/OR ACRYLIC ACID USING THE CATALYST

(76) Inventors: Michio Tanimoto, Himeji-shi (JP); Nobuyuki Hakozaki, Himeji-shi (JP)

(21) Appl. No.: 13/003,116

(22) PCT Filed: Sep. 25, 2009
(86) PCT No.: PCT/JP2009/066657

§ 371 Date: Jan. 7, 2011

(30) Foreign Application Priority Data
Sep. 30, 2008 (JP) 2008-252084

(51) Int. Cl. C07C 51/25; B01J 23/887; C07C 45/35

(52) U.S. Cl. 562/546; 502/311; 568/479

(57) ABSTRACT

A catalyst for producing acrolein and/or acrylic acid from propylene by gas-phase catalytic oxidation with molecular oxygen-containing gas, comprising a catalytic active component containing molybdenum, bismuth and cobalt as essential elements, and having a crystallinity T of 4% or more and 18% or less in the 2θ range of 5° to 90°, measured by an X-ray diffraction analysis with Cu—Kα radiation; and a process for producing acrolein and/or acrylic acid by gas-phase catalytic oxidation of propylene with molecular oxygen, comprising the step of conducting the gas-phase catalytic oxidation in the presence of the catalyst. According to the catalyst and the process for producing acrolein and/or acrylic acid of the present invention, it is possible to stably produce acrolein and/or acrylic acid at a high yield for a long period.

### TECHNICAL FIELD

The present invention relates to a catalyst suitable for producing acrolein and/or acrylic acid from propylene by gas-phase catalytic oxidation with molecular oxygen and a process for producing acrolein and/or acrylic acid using the catalyst.

### BACKGROUND ART

Acrylic acid is industrially important as a raw material for various synthetic resins, paints and plasticizers, and recently, its importance as a raw material for an absorbent polymer has been increasing. The most common process for producing acrylic acid is a two-stage oxidation process in which acrolein is mainly produced from propylene by gas-phase catalytic oxidation and then acrylic acid is produced from the obtained acrolein by gas-phase catalytic oxidation.

Upon the process for producing acrylic acid by gas-phase catalytic oxidation of propylene in the presence of molecular oxygen-containing gas, an oxidation catalyst is used for producing acrolein and/or acrylic acid from propylene in the above first stage. However, the catalytic performance such as an yield of acrolein, catalytic life and the like, of this catalyst is not necessarily satisfactory, and various modifications centered around a molybdenum-bismuth-containing catalyst have been made by many companies for the purpose of improving catalyst performance.

For example, Patent literature 1 discloses a catalyst which has crystalline phases of β-X¹MoO₄ mainly and Fe₂(MoO₄)₃ secondly and does not contain MoO₃. Patent literature 2 discloses a catalyst consisting of cobalt, molybdenum, bismuth and iron, and having a crystalline phase expressed by Bi₂Fe₂Mo₂O₁₂ in its active component. Patent literature 3 discloses a method for preparing a catalyst containing molybdenum, bismuth and iron as essential elements, in which dry-powdered iron molybdate gel (Fe(MoO₄)₃) is used for preparing the catalyst. Patent literature 4 discloses that, in a catalyst containing molybdenum, bismuth and iron as essential elements, by heating the catalyst with reduced catalytic activity under specific conditions, a peak attributed to a crystalline phase Bi₂Fe₂Mo₂O₁₂ in a catalytic active component reduces whereas a peak attributed to Fe₂(MoO₄)₃ is regrown, resulting in activation of the catalyst performance.

### CITATION LIST

### Patent Literature

### Patent Literature 1

Japanese Unexamined Patent Application Publication No. 2000-169149

### Patent Literature 2

Japanese Examined Patent Application Publication No. 56-28180

### Patent Literature 3

Japanese Unexamined Patent Application Publication No. 1-168344

### Patent Literature 4

Japanese Unexamined Patent Application Publication No. 63-137755

### SUMMARY OF INVENTION

### Technical Problem

Acrylic acid is produced as much as several millions of tons per year worldwide, and in addition, the demand therefor as a raw material for an absorbent polymer is still increasing. Further, due to recent jumps in prices of the raw material propylene, enhancing the yield of acrylic acid in an industrial scale by just 0.1% would have a significant meaning in terms of economy. Upon the process for producing acrolein and/or acrylic acid from propylene, that is the above first stage, enhancement of its yield is desired. Although the catalysts disclosed in Patent literatures 1 to 4 are somewhat improved in the intended catalyst performance such as the yield of acrolein and/or acrylic acid, catalyst life and the like, there is still room for improvement in an industrial scale.

In the catalyst disclosed in Patent literature 1, a space-time yield of acrolein and acrylic acid is relatively high; however, it is evaluated under a condition of a low raw material propylene concentration of 5.5%. Further, even under the condition where the raw material propylene concentration is low, the reaction temperature must be increased by as much as 10° C. after only 2000 hours of operation, and thus, the suppression of performance deterioration with time is insufficient and there is unknown about the catalyst performance when the reaction is continued for a long period. The catalyst described in the Patent literature 2 has an acrylic acid selectivity of at most about 88% in an initial stage of the reaction, which is insufficient in respect of the yield of acrylic acid. In addition, the catalyst life is not evaluated and there is unknown about the catalyst performance when acrylic acid is continuously produced over a long period. In Example of Patent literature 3, though it is disclosed that the yield of acrylic acid is improved in some extent, it is a result of being reacted in a laboratory-scale reaction tube under a condition of a low raw material propylene concentration of 4.5%. In addition, the catalyst life is not evaluated and there is unknown about the practical catalyst performance. Patent literature 4 merely discloses a method for reactivating of a well-known catalyst and does not teach a new catalyst for the purpose of improving the catalyst performance. Furthermore, none of Patent literatures 1 to 4 discloses a technical idea of enhancing the yields of acrolein and/or acrylic acid over a long period by controlling a crystallinity of a catalytic active component within an appropriate range.

The present invention has been achieved in view of the above circumstances, and an object of the present invention is to provide a catalyst which is excellent in catalyst performance such as catalytic activity and selectivity as well as in catalyst life and provides stable performance over a long period in producing acrolein and/or acrylic acid from propylene.

### Solution to Problem

The catalyst of the present invention which solves the above problems is a catalyst for producing acrolein and/or acrylic acid from propylene by gas-phase catalytic oxidation with molecular oxygen-containing gas, comprising: a catalytic active component containing molybdenum, bismuth and cobalt as essential elements, and having a crystallinity T of 4% or more and 18% or less in the 2θ range of 5° to 90°, measured by an X-ray diffraction analysis with Cu—Kα radiation. According to the catalyst of the present invention, since the crystallinity T is within the aforementioned range, the catalyst is excellent in catalytic performance such as catalytic activity and selectivity as well as in catalyst life, and provides stable performance over a long period.

In the catalyst of the present invention, it is preferred that a crystallinity ratio R, which is calculated as a ratio of a crystallinity M of the peak at 2θ=26.5±0.5° in the catalytic active component, measured by the X-ray diffraction analysis with Cu—Kα radiation, to the crystallinity T, as expressed by the following formula (1), is preferably in the range of 0.03 or more and 0.20 or less. When the crystallinity ratio R is adjusted in that range in addition to the crystallinity T, it becomes easy to obtain a catalyst for producing acrolein and/or acrylic acid which is more excellent in catalyst performance and catalyst life and provides stable performance over a long period.

R=M/T (1)

In addition, the process for producing acrolein and/or acrylic acid of the present invention is a process for producing acrolein and/or acrylic acid by gas-phase catalytic oxidation of propylene with molecular oxygen-containing gas, comprising the step of conducting the gas-phase catalytic oxidation in the presence of the catalyst of the present invention. According to the process of the present invention, it is possible to stably produce acrolein and/or acrylic acid at a high yield for a long period.

### ADVANTAGEOUS EFFECTS OF INVENTION

According to the catalyst and the process for producing acrolein and/or acrylic acid of the present invention, it is possible to stably produce acrolein and/or acrylic acid at a high yield for a long period in producing acrolein and/or acrylic acid by gas-phase catalytic oxidation of propylene with molecular oxygen.

### DESCRIPTION OF EMBODIMENTS

The catalyst for producing acrolein and/or acrylic acid of the present invention and the process for producing acrolein and/or acrylic acid of the present invention using the catalyst will hereinafter be explained in detail; however, the present invention is not limited to the following description, and can be put into practice after appropriate modification or variations within a range meeting the gist of the present invention in addition to the following embodiments.

The catalyst for producing acrolein and/or acrylic acid of the present invention is a catalyst for producing acrolein and/or acrylic acid by gas-phase catalytic oxidation of propylene with molecular oxygen-containing gas. A raw material gas for producing acrolein and/or acrylic acid contains at least propylene and molecular oxygen-containing gas. The molecular oxygen-containing gas is not particularly limited as long as it contains molecular oxygen, and for example, gas consisting of only molecular oxygen may be used, or air may be used, as the molecular oxygen-containing gas.

The catalyst of the present invention comprises a catalytic active component containing molybdenum, bismuth and cobalt as essential elements, and the catalytic active component has a crystallinity T of 4% or more and 18% or less in the 2θ range of 5° to 90°, measured by an X-ray diffraction analysis with Cu—Kα radiation. The crystallinity T is preferably 7% or more and 15% or less. In the catalyst of the present invention, since the catalytic active component has the crystallinity T of 4% or more and 18% or less in the 2θ range of 5° to 90°, measured by an X-ray diffraction analysis with Cu—Kα radiation, the catalyst is excellent in catalytic performance such as catalytic activity, selectivity and the like, as well as in catalyst life, and provides stable performance over a long period. Although the reason is not clear, when the crystallinity T is less than 4%, the catalyst has low selectivity for producing acrolein and/or acrylic acid, whereas when the crystallinity T is more than 18%, the catalyst life becomes short, resulting in early degradation in performance.

The crystallinity in the present invention can be obtained by using an X-ray diffraction apparatus following the method according to JIS K-0131 (an absolute method). Thus, the crystallinity T is calculated by obtaining an X-ray diffraction intensity of all crystalline part(s) in the 2θ range of 5° to 90° and a total X-ray diffraction intensity in the 2θ range of 5° to 90°, and obtaining the ratio of the X-ray diffraction intensity of the all crystalline part(s) to the total X-ray diffraction intensity, as expressed by the following formula (2). Here, the total X-ray diffraction intensity means an integral intensity of the X-ray diffraction in the 2θ range of 5° to 90°, and the X-ray diffraction intensity of the all crystalline part(s) means an integral intensity of the X-ray diffraction of the crystalline part(s) in the 2θ range of 5° to 90°.

T=(an X-ray diffraction intensity of the all crystalline part(s))/(a total X-ray diffraction intensity)×100 (2)

The crystallinity T is obtained as follows, specifically. From a diffraction profile obtained by the X-ray diffraction analysis, a background is eliminated and then a halo pattern of an amorphous part is separated, thereby obtaining a diffraction profile of crystalline part(s). The diffraction profile of the crystalline part(s) is separated by peaks. An integral intensity (Sₙ) of the halo pattern of the amorphous part and integral intensities (S_{c1}, S_{c2}, S_{c3} . . . ) of the respective peaks of the crystalline part(s) are obtained, and the X-ray diffraction intensity S_{c} of all the crystalline part(s) is calculated from the sum of the integral intensities (S_{c1}+S_{c2}+S_{c3}+ . . . ) of the respective peaks of the crystalline part(s). The crystallinity T is obtained from the formula: T=S_{c}/(Sₙ+S_{c})×100.

In the catalyst of the present invention, a crystallinity ratio R, which is calculated as a ratio of a crystallinity M of the peak at 2θ=26.5±0.5° in the catalytic active component, measured by the X-ray diffraction analysis with Cu—Kα radiation, to the crystallinity T, as expressed by the following formula (1), is preferably in the range of 0.03 or more and 0.20 or less. More preferably, the crystallinity ratio is in the range of 0.05 or more and 0.13 or less.

R=M/T (1)

In the above formula (1), M represents a crystallinity of the peak at 2θ=26.5±0.5° when the catalytic active component is measured by the X-ray diffraction analysis with Cu—Kα radiation. The M can be obtained by using an X-ray diffraction apparatus following the method according to JIS K-0131 (the absolute method). Thus, the M is calculated by obtaining an X-ray diffraction intensity of a crystalline part of the peak at 2θ=26.5±0.5° and a total X-ray diffraction intensity in the 2θ range of 5° to 90°, and obtaining the ratio of the X-ray diffraction intensity at 2θ=26.5±0.5° to the total X-ray diffraction intensity, as expressed by the following formula (3). Here, the X-ray diffraction intensity of the crystalline part of the peak at 2θ=26.5±0.5° means an integral intensity of the X-ray diffraction having a peak at 2θ=26.5±0.5°.

M=(an X-ray diffraction intensity of a crystalline part of the peak)/(a total X-ray diffraction intensity)×100 (3)

The crystallinity ratio R corresponds to the ratio of the X-ray diffraction intensity of the crystalline part of the peak at 2θ=26.5±0.5° to the X-ray diffraction intensity of all the crystalline part(s) in the 2θ range of 5° to 90°. Thus, by defining the crystallinity ratio R, the ratio of the specific crystalline part in the whole crystalline part is defined, whereby a catalyst for producing acrylic acid which is more excellent in catalyst performance and catalyst life and provides stable performance over a long period can be easily obtained.

The catalyst for producing acrolein and/or acrylic acid of the present invention comprises the catalytic active component containing molybdenum, bismuth and cobalt as essential elements. For the catalyst for producing acrolein and/or acrylic acid of the present invention, it is important that the crystallinity T is within the aforementioned range, and the present invention can be applied to a catalyst which contains molybdenum, bismuth and cobalt essentially as the catalytic active component. As to the catalyst which contains molybdenum, bismuth and cobalt as essential elements, compositions of the catalytic active component and preparing methods are variously proposed by many companies; however, in the present invention, it is preferred that the catalyst comprises the catalytic active component having the composition expressed by the following formula (4). When the catalyst comprises the catalytic active component having the composition expressed by the following formula (4), the catalyst which is excellent in catalyst performance such as activity (i.e. conversion rate of propylene and yields of acrolein and/or acrylic acid), catalyst life and the like is easily obtained by adjusting the crystallinity T in an appropriate range:

MoₐBi_{b}Co_{c}X1_{d}X2ₑX3_{f}X4_{g}O_{z} (4)

wherein Mo is molybdenum; Bi is bismuth; Co is cobalt; X1 represents at least one kind of element selected from the group consisting of iron and nickel; X2 represents at least one kind of element selected from the group consisting of alkali metals, alkali earth metals, boron and thallium; X3 represents at least one kind of element selected from the group consisting of tungsten, silicon, aluminum, zirconium and titanium; X4 represents at least one kind of element selected from the group consisting of phosphorus, tellurium, antimony, tin, cerium, lead, niobium, manganese, arsenic and zinc; O is oxygen; a, b, c, d, e, f, g and z mean atomic ratios of Mo, Bi, Co, X1, X2, X3, X4 and 0, respectively, and meet inequalities: a=12, 0.1≦b≦10, 0.1≦c≦20, 0.1≦d≦20, 0.001≦e≦10, 0≦f≦30 and 0≦g≦10, respectively; and z is a numeral value determined by oxidation states of respective elements.

The catalyst of the present invention may comprise an inert carrier for supporting the catalytic active component in addition to the catalytic component containing molybdenum, bismuth and cobalt as essential elements. Examples of the inert carrier include alumina, silica, silica-alumina, titania, magnesia, steatite, cordierite, silica-magnesia, silicon carbide, silicon nitride, zeolite, and the like. A shape of the carrier is not particularly limited, and a carrier having a spherical shape, a cylindrical shape, a ring shape, or the like, may be used.

A preparing method of the catalyst of the present invention is hereinafter explained. The preparing method of a catalyst is variously proposed; and the following method may be employed, for example.

As a raw material for the catalytic active component, an oxide, a hydroxide, or a salt (e.g. an ammonium salt, a nitrate salt, a carbonate salt, a sulfate salt, an organic acid salt or the like) of each element composing the catalytic active component, an aqueous solution or a sol thereof, a compound containing a plurality of elements, or the like, is mixed with water to give an aqueous solution or an aqueous slurry (hereinafter referred to as a “mixed liquid of starting materials”), for example.

The obtained mixed liquid of starting materials is dried by a method such as heating or pressure reduction as required to obtain a catalyst precursor. For the drying method by heating, for example, a spray dryer or a drum dryer may be used, resulting in obtaining a powdery catalyst precursor. Alternatively, the mixed liquid of starting materials may be heated using a box dryer, a tunnel dryer or the like, while flowing an air, to obtain a blockish or flaky catalyst precursor. Furthermore, it is also possible to employ a method of concentrating and evaporating the mixed liquid of starting materials to dryness to give a cake-like solid matter and further heating the solid matter as described above. For the drying method by pressure reduction, for example, a vacuum dryer may be used, thereby obtaining a blockish or flaky catalyst precursor.

The obtained catalyst precursor may be gone through a pulverization step and a classification step for giving a powder having an appropriate particle size, as required, and then sent to a subsequent forming step. In some cases, the catalyst precursor may be once calcinated and then sent to the forming step. A particle size of the catalyst precursor before being sent to the forming step is not particularly limited; however, the catalyst precursor preferably has a particle size such that 90 mass % or more of the particles pass though a sieve with a mesh size of 500 μm, in view of obtaining excellent formability.

In the forming step, a compact may be obtained by forming the catalyst precursor into a specific shape by extrusion molding, tablet compression, or the like. Alternatively, a supported catalyst may be obtained by making any inert carrier having a specific shape support the mixed liquid of starting materials or the catalyst precursor as the catalytic active component (supporting method).

A shape of the compact obtained by extrusion molding, tablet compression or the like is not particularly limited, and any shape such as a spherical shape, a cylindrical shape, a ring shape, an indeterminate shape or the like, may be taken. Of course, the spherical shape does not have to be a true sphere and a substantially spherical shape will suffice. The same applies also to the cylindrical shape and the ring shape.

As the supporting method, an evaporation to dryness method in which the mixed liquid of starting materials, which is not dried and remains in the form of an aqueous solution or an aqueous slurry, is applied or attached to an inert carrier having a specific shape while heating, and then dried to make the inert carrier support the mixed liquid of starting materials, may be employed, or a granulation method in which an inert carrier is made to support a powdery catalyst precursor may be employed. Among them, the granulation method for making an inert carrier support the catalytic active component with using a centrifugal fluidizing coating method described in Japanese Unexamined Patent Application Publication No. 63-200839, a tumbling granulation method described in Japanese Unexamined Patent Application Publication No. 8-299797, or a rocking mixer method described in Japanese Unexamined Patent Application Publication No. 2004-136267, is particularly preferred. A material and shape of the inert carrier which can be used for the supporting method is explained above.

In the forming step, a forming auxiliary agent for enhancing moldability, a binder, a pore-forming agent for forming appropriate pores in a catalyst, or the like may be added. Specific examples of these agents include organic compounds such as ethylene glycol, glycerin, propionic acid, maleic acid, benzyl alcohol, propyl alcohol, butyl alcohol, phenol and the like; water; and inorganic salts such as nitric acid, ammonium nitrate, ammonium carbonate and the like.

In addition, a strengthening agent such as silica, alumina, glass fiber, silicon carbide, silicon nitride and the like can be used for the purpose of enhancing mechanical strength of the catalyst. The strengthening agent may be added to the mixed liquid of starting materials or may be mixed with the catalyst precursor.

The compact or the supported catalyst which have been obtained in the forming step is sent to a subsequent calcination step, where the compact or the supported catalyst is calcinated. The calcination temperature is preferably in the range of 350° C. or more and 600° C. or less, and more preferably in the range of 400° C. or more and 550° C. or less. The calcination period is preferably 1 hour or longer and 10 hours or shorter. A calcinating furnace used for the calcination step is not restricted, and a box furnace, a tunnel furnace or the like, which are generally used, may be used.

In the catalyst of the present invention, the crystallinity T is within the specific range, and further, the crystallinity ratio R is preferably within the specific range. For obtaining such a catalyst, it is preferred to adopt the following method. Thus, in the above preparation method of a catalyst, upon preparation of the mixed liquid of starting materials, the raw material may be fed in portions, or the feeding period of the raw material or the temperature of the mixed liquid upon mixing of the raw materials may be adjusted appropriately; or the catalyst which has been calcinated may be subjected to a hydrothermal treatment under specific conditions. Specifically, when preparing the mixed liquid of starting materials, the raw material such as molybdenum, bismuth, cobalt and the like may be fed in two or more portions; or when adding an aqueous solution (solution B) containing cobalt, bismuth and the like to an aqueous solution (solution A) containing molybdenum and the like, the solution B may be added over a period of 30 seconds to 10 minutes, preferably 1 minute to 5 minutes. The temperature of the mixed liquid upon mixing the raw materials may be appropriately determined according to the raw materials to be fed. In the case that the catalyst is subjected to a hydrothermal treatment under specific conditions, the catalyst which has been calcinated may be put in an autoclave with water serving as saturated water vapor therein, and treated for 2 to 48 hours under pressure at a temperature in the range of 150° C. to 250° C., preferably 190° C. to 240° C. In the above method, it is possible to change the crystallinity ratio R by appropriately varying the composition ratio among molybdenum, bismuth and cobalt in the catalytic active component.

A process for producing acrolein and/or acrylic acid of the present invention is hereinafter explained. The process for producing acrolein and/or acrylic acid of the present invention is a process for producing acrolein and/or acrylic acid by gas-phase catalytic oxidation of propylene with molecular oxygen-containing gas, comprising the step of conducting the gas-phase catalytic oxidation in the presence of the catalyst of the present invention. According to the process for producing acrolein and/or acrylic acid of the present invention, it is possible to stably produce acrolein and/or acrylic acid at a high yield for a long period.

In the process for producing acrolein and/or acrylic acid of the present invention, a reactor to be used is not restricted as long as gas-phase catalytic oxidation is conducted by using the catalyst of the present invention. Any reactor such as a fixed-bed reactor, a fluidized-bed reactor, and a moving bed reactor can be used as the reactor. Preferably, a fixed-bed reactor is used as the reactor.

As the fixed-bed reactor, a tubular reactor is preferable and a multitubular reactor is more preferable. In this case, the fixed-bed reactor comprises one or more of reaction tube(s), in which the catalyst is filled. The reaction tube is generally placed so as to be vertically-oriented in the reactor. An inner diameter of the reaction tube is not particularly limited as long as the catalyst can be filled therein; however, it is preferably in the range of 15 mm or more and 50 mm or less, more preferably 20 mm or more and 40 mm or less, and even more preferably 22 mm or more and 38 mm or less.

The catalyst to be filled in the reaction tube may be one kind or may be two or more kinds; however, the catalyst is preferably filled in the reaction tube preferably to define a plurality of reaction zones, which are provided by dividing the catalyst layer filled in the reaction tube into two or more zones in an axis direction of the reaction tube, such that the catalysts filled in the respective reaction zones have the different crystallinity ratio R from each other. Thus, it is preferable that the reaction tube has a plurality of reaction zones divided in the axis direction of the reaction tube, and the respective reaction zones are filled with the catalysts having the different crystallinity ratio R from each other. More preferably, the catalyst is filled in the reaction tube such that the crystallinity ratios R of the plurality of the reaction zones decrease in an order from an inlet to an outlet of a raw material gas containing propylene and molecular oxygen-containing gas. In this case, the catalysts filled in the respective reaction zones preferably have the crystallinity ratio R in the range of 0.03 or more and 0.20 or less. Further, the above catalyst filling manner may be combined with the following manner: the catalyst is filled such that the volume occupied by the catalyst decreases from the inlet to the outlet of the raw material gas; or a part of the catalyst is diluted with an inert carrier or the like.

A number of the reaction zone is determined appropriately depending on reaction conditions or a scale of the reactor. However, from an industrial viewpoint, the number of the reaction zones is preferably about 2 or more and about 6 or less, because the excessively large number of the reaction zones will cause problems such as complication of a filling operation of the catalyst.

Reaction conditions in the process for producing acrolein and/or acrylic acid of the present invention is not particularly limited; and any reaction condition generally used in this kind of reaction can be adopted. For example, a mixed gas, composed of 1 to 15 volume % (preferably, 4 to 12 volume %) of propylene, 0.5 to 25 volume % (preferably, 2 to 20 volume %) of molecular oxygen, 0 to 30 volume % (preferably, 0 to 25 volume %) of steam, and inert gas such as nitrogen and the like as the residue, may be used as the raw material gas. The raw material gas may be brought into contact with the catalyst at a temperature in the range of 280° C. to 450° C., under a pressure in the range of 0.1 MPa to 1.0 MPa, at a space velocity (STP) in the range of 300 h⁻¹ to 5,000 h⁻¹.

### EXAMPLES

The present invention will hereinafter be described more specifically by reference to Examples; however, the present invention is not limited to these Examples. The present invention can be put into practice after appropriate modifications or variations within a range meeting the gist of the present invention, all of which are included in the technical scope of the present invention. Hereinafter, the term “part(s) by mass” may be described simply as “part(s)” for convenience sake.

Conversion rate of propylene and a sum of yields of acrolein and acrylic acid were determined by the following equations:

Conversion rate of propylene (mol %)=(molar quantity of reacted propylene)/(molar quantity of fed propylene)×100

Yield of acrolein+acrylic acid (mol %)=(molar quantities of produced acrolein and acrylic acid)/(molar quantity of fed propylene)×100

An X-ray diffraction measurement was conducted using X'PertPro manufactured by Spectris Co., Ltd. A sample for the X-ray diffraction measurement was produced by passing a catalytic active component through a sieve with a mesh size of 150 μm and compression-forming about 0.5 g of the powder that passed through the sieve into tablets each having a diameter of 16 mm and a thickness of 2 mm. The sample for the X-ray diffraction measurement was used to measure the X-ray diffraction intensity with Cu—Kα radiation in the 2θ range of 5° to 90° under conditions of an X-ray output of 40 mA and 45 kV, an X-ray radiation width of 15 mm, and a measurement temperature of 25° C. From the obtained X-ray diffraction profile, the crystallinity T and the crystallinity ratio R were obtained, following the method according to JIS K-0131 (an absolute method).

(1) Experiment Example 1

(1-1) Preparation of Catalyst

3000 parts of distilled water was stirred with the temperature maintained at 60° C., and 300 parts-of ammonium paramolybdate, and 1.7 parts of potassium nitrate were dissolved therein to give a solution A. Separately, 300 parts of distilled water was stirred with the temperature maintained at 60° C., and 50 parts of 65 mass % of nitric acid, 137 parts of bismuth nitrate, 412 parts of cobalt nitrate, 114 parts of iron nitrate and 206 parts of nickel nitrate were added thereto to give a solution B. The obtained solution B were divided into two so as to be the ratio of 1:1 by mass part to give a solutions B-1 and B-2. While the solution A being stirred, the solution B-1 was added to the solution A over 2 minutes, and the obtained mixture was stirred for 30 minutes. Subsequently, the solution B-2 was added thereto over 2 minutes, and then stirred for 30 minutes to give a suspended liquid. 200 parts of ammonium paramolybdate was further added to the suspension liquid, and the obtained mixture was stirred for 30 minutes to give a mixed liquid of starting materials. The mixed liquid of starting materials was evaporated to give a cake-like solid matter, and the solid matter was dried for about 5 hours at 200° C. under an air atmosphere. The dried solid matter was pulverized to a particle diameter of 250 μm or less to give a powdery catalyst precursor. Into a tumbling granulator, 500 parts of a silica-alumina spherical carrier having an average particle diameter of 4.5 mm was fed and then the catalyst precursor was fed together with a 15 mass % ammonium nitrate aqueous solution as a binder to make the carrier support the catalyst precursor. Then, the obtained supported catalyst precursor was calcinated at 470° C. for 6 hours under an air atmosphere to give a catalyst 1. The supported ratio of the catalyst 1 was 100 mass %, and the catalyst 1 had the following metal element composition excluding oxygen.

### Catalyst 1: Mo₁₂Bi₁₂Co₆Fe_{1.2}Ni₃K_{0.07}

The supported ratio was determined by the following equations:

Supported ratio (mass %)=(mass of a catalyst (g)−mass of a used carrier (g))/(mass of a used carrier (g))×100

In the catalyst 1, the crystallinity T in the 20 range of 5° to 90°, measured by an X-ray diffraction analysis, of the catalytic active component was 7.1%, and the crystallinity ratio R was 0.10.

(1-2) Reaction

A reactor having a reaction tube (reaction tube length: 3,000 mm, inner diameter: 25 mm) made of stainless steel and a shell in which a heat medium is flowed and which covers the reaction tube was vertically-installed. The catalyst 1 was filled into the reactor by dropping the catalyst 1 from the top of the reaction tube so that the layer length comes to be 2900 mm. While maintaining the temperature of the heat medium (a reaction temperature) at 310° C., a mixed gas consisting of 6 volume % of propylene, 11 volume % of oxygen, 15 volume % of steam and an inert gas such as nitrogen as the residue was introduced from the bottom of the reaction tube filled with the catalyst at a space velocity (STP) of 1650 hr⁻¹, thereby conducting oxidation reaction of propylene. The results of the conversion rate of propylene and the yield of acrolein+acrylic acid are shown in Table 1. Further, the result after continuous reaction for 4000 hours, that was conducted while adjusting the reaction temperature appropriately, is also shown in Table 1. In Experiment Example 1, the initial yield was as high as 92.8 mol %, and the decrease of the yield after 4000 hours was suppressed to be only in the amount of 0.2 mol %. In Experiment Example 1, acrolein and acrylic acid were produced stably at a high yield for a long period.

(2) Experiment Example 2

A catalyst 2 was prepared in the same manner as in Experiment Example 1, except that the maintaining temperature of the solutions A and B were changed to 70° C., the amounts of ammonium paramolybdate added to the solution A and the suspension liquid were changed to 500 parts and 0 part, respectively, the solution B was not divided into two, and the period for adding the solution B to the solution A was changed to within 10 seconds in Experiment Example 1. The supported ratio of the catalyst 2 was 100 mass %, and the metal element composition of the catalyst 2 was the same as that of the catalyst 1. In the catalyst 2, the crystallinity T was 18.9% and the crystallinity ratio R was 0.02.

The obtained catalyst 2 was filled in the same manner as in Experiment Example 1 and oxidation reaction of propylene was conducted under the same conditions as in Experiment Example 1. The results are shown in Table 1. Further, the result after continuous reaction for 4000 hours, that was conducted while adjusting the reaction temperature appropriately, is also shown in Table 1. In Experiment Example 2, since the crystallinity T of the catalyst 2 was more than 18% and the crystallinity ratio R of the catalyst 2 was a low value of 0.02, the initial yield was as low as 90.7 mol %. After 4000 hours, the yield further decreased, and the amount of the decrease of the yield was as much as 1.5 mol % compared to the initial stage.

(3) Experiment Example 3

A catalyst 3 was prepared in the same manner as in Experiment Example 1, except that the maintaining temperature of the solutions A and B were changed to 70° C., the amounts of ammonium paramolybdate added to the solution A and the suspension liquid were changed to 500 parts and 0 part, respectively, the amounts of bismuth nitrate, cobalt nitrate, iron nitrate and nickel nitrate were changed to 206 parts, 481 parts, 191 parts and 103 parts, respectively, and the periods for adding the solutions B-1 and B-2 to the solution A were respectively changed to 1 minute in Experiment Example 1. The supported ratio of the catalyst 3 was 100 mass %, and the catalyst 3 had the following metal element composition excluding oxygen.

### Catalyst 3: Mo₁₂Bi_{1.8}Co₇Fe₂Ni_{1.5}K_{0.07}

In the catalyst 3, the crystallinity T was 14.3% and the crystallinity ratio R was 0.06.

The obtained catalyst 3 was filled in the same manner as in Experiment Example 1 and oxidation reaction of propylene was conducted under the same conditions as in Experiment Example 1. The results are shown in Table 1. In Experiment Example 3, the initial yield was as high as 93.0 mol %.

(4) Experiment Example 4

A catalyst 4 was prepared in the same manner as in Experiment Example 1, except that the amounts of ammonium paramolybdate added to the solution A and the suspension liquid were changed to 100 parts and 400 parts, respectively, the amounts of bismuth nitrate, cobalt nitrate, nickel nitrate and potassium nitrate were changed to 172 parts, 343 parts, 137 parts and 1.2 parts, respectively, the solution B was not divided into two, the period for adding the solution B to the solution A was changed to 12 minutes, and the amount of silica-alumina spherical carrier fed into the tumbling granulator was changed to 450 parts in Experiment Example 1. The supported ratio of the catalyst 4 was 100 mass %, and the catalyst 4 had the following metal element composition excluding oxygen.

### Catalyst 4: Mo₁₂Bi_{1.5}Co₅Fe_{1.2}Ni₂K_{0.05}

In the catalyst 4, the crystallinity T was 4.5% and the crystallinity ratio R was 0.21.

The obtained catalyst 4 was filled in the same manner as in Experiment Example 1 and oxidation reaction of propylene was conducted under the same conditions as in Experiment Example 1. The results are shown in Table 1. Further, the result after continuous reaction for 4000 hours, that was conducted while adjusting the reaction temperature appropriately, is also shown in Table 1. In Experiment Example 4, since the catalyst 4 had a somewhat low crystallinity T of 4.5% and a high crystallinity ratio R of 0.21, the yield was lower than those in Experiment Examples 1 and 3, that is, 91.7 mol % in the initial stage and 90.9 mol % after 4000 hours.

(5) Experiment Example 5

A catalyst 5 was prepared in the same manner as in Experiment Example 4, except that the amounts of ammonium paramolybdate added to the solution A and the suspension liquid were changed to 0 part and 500 parts, respectively, and the period for adding the solution B to the solution A was changed to 20 minutes in Experiment Example 4. The supported ratio of the catalyst 5 was 100 mass %, and the metal element composition of the catalyst 5 was the same as that of the catalyst 4. In the catalyst 5, the crystallinity T was 3.4% and the crystallinity ratio R was 0.22.

The obtained catalyst 5 was filled in the same manner as in Experiment Example 1 and oxidation reaction of propylene was conducted under the same conditions as in Experiment Example 1. The results are shown in Table 1. Further, the result after continuous reaction for 4000 hours, that was conducted while adjusting the reaction temperature appropriately, is also shown in Table 1. In Experiment Example 5, since the crystallinity T of the catalyst 5 was less than 4% and the crystallinity ratio R of the catalyst 5 was as high as 0.22, the initial yield was resulted in a low value of 89.6 mol %. After 4000 hours, the yield further decreased, and the amount of the decrease of the yield was as much as 1.9 mol % compared to the initial stage.

(6) Experiment Example 6

A catalyst 6 was prepared in the same manner as in Experiment Example 3, except that the amounts of bismuth nitrate and iron nitrate were changed to 183 parts and 114 parts, respectively, and the periods for adding the solutions B-1 and B-2 to the solution A were respectively changed to within 10 seconds in Experiment Example 3. The supported ratio of the catalyst 6 was 100 mass %, and the catalyst 6 had the following metal element composition excluding oxygen.

### Catalyst 6: Mo₁₂Bi_{1.6}Co₇Fe_{1.2}Ni_{1.5}K_{0.07}

In the catalyst 6, the crystallinity T was 17.1% and the crystallinity ratio R was 0.02.

The obtained catalyst 6 was filled in the same manner as in Experiment Example 1 and oxidation reaction of propylene was conducted under the same conditions as in Experiment Example 1. The results are shown in Table 1. In Experiment Example 6, since the catalyst 6 had a somewhat high crystallinity T of 17.1% and a low crystallinity ratio R of 0.02, the initial yield was lower than those in Experiment Examples 1 and 3, that is, 91.4 mol %.

(7) Experiment Example 7

A catalyst 7 was prepared in the same manner as in Experiment Example 6, except that the maintaining temperature of the solutions A and B were changed to 50° C., the amounts of ammonium paramolybdate added to the solution A and the suspension liquid were changed to 0 part and 500 parts, respectively, and the solution B was not divided into two in Experiment Example 6. The supported ratio of the catalyst 7 was 100 mass %, and the metal element composition of the catalyst 7 was the same as that of the catalyst 6. In the catalyst 7, the crystallinity T was 3.4% and the crystallinity ratio R was 0.01.

The obtained catalyst 7 was filled in the same manner as in Experiment Example 1 and oxidation reaction of propylene was conducted under the same conditions as in Experiment Example 1. The results are shown in Table 1. In Experiment Example 7, since the crystallinity T of the catalyst 7 was less than 4% and the crystallinity ratio R was as low as 0.01, the initial yield was resulted in a low value of 90.4 mol %.

(8) Experiment Example 8

A catalyst 8 was prepared in the same manner as in Experiment Example 4, except that the maintaining temperature of the solutions A and B were changed to 70° C., the amounts of ammonium paramolybdate added to the solution A and the suspension liquid were changed to 400 parts and 100 parts, respectively, the amounts of bismuth nitrate and iron nitrate were changed to 145 parts and 95.4 parts, respectively, nickel nitrate was not added, the period for adding the solution B to the solution A was changed to 30 seconds, and the amount of silica-alumina spherical carrier fed into the tumbling granulator was changed to 430 parts in Experiment Example 4. The supported ratio of the catalyst 8 was 100 mass %, and the catalyst 8 had the following metal element composition excluding oxygen.

### Catalyst 8: Mo₁₂Bi_{1.3}Co₅Fe₁K_{0.05}

In the catalyst 8, the crystallinity T was 9.7% and the crystallinity ratio R was 0.04.

The obtained catalyst 8 was filled in the same manner as in Experiment

Example 1 and oxidation reaction of propylene was conducted under the same conditions as in Experiment Example 1. The results are shown in Table 1. In Experiment Example 8, the initial yield was as high as 92.6 mol %.

(9) Experiment Example 9

A catalyst 9 was prepared in the same manner as in Experiment Example 1, except that the maintaining temperature of the solutions A and B were changed to 70° C., the amounts of ammonium paramolybdate added to the solution A and the suspension liquid were changed to 350 parts and 150 parts, respectively, the amounts of bismuth nitrate, cobalt nitrate and iron nitrate were changed to 229 parts, 481 parts and 143 parts, nickel nitrate was not added, and the periods for adding the solutions B-1 and B-2 to the solution A were respectively changed to 1 minute in Experiment Example 1. The supported ratio of the catalyst 9 was 100 mass %, and the catalyst 9 had the following metal element composition excluding oxygen.

### Catalyst 9: Mo₁₂Bi₂Co₇Fe_{1.5}K_{0.07}

In the catalyst 9, the crystallinity T was 7.9% and the crystallinity ratio R was 0.12.

The obtained catalyst 9 was filled in the same manner as in Experiment Example 1 and oxidation reaction of propylene was conducted under the same conditions as in Experiment Example 1. The results are shown in Table 1. In Experiment Example 9, the initial yield was as high as 93.0 mol %.

(10) Experiment Example 10

A catalyst 10 was prepared in the same manner as in Experiment Example 1, except that the amounts of ammonium paramolybdate added to the solution A and the suspension liquid were changed to 0 part and 500 parts, respectively, the amounts of bismuth nitrate, cobalt nitrate, iron nitrate and potassium nitrate were changed to 80.1 parts, 378 parts, 143 parts and 1.2 parts, nickel nitrate was not added, the periods for adding the solutions B-1 and B-2 to the solution A were respectively changed to 4 minutes, and the amount of silica-alumina spherical carrier fed into the tumbling granulator was changed to 430 parts in Experiment Example 1. The supported ratio of the catalyst 10 was 100 mass %, and the catalyst 10 had the following metal element composition excluding oxygen.

### Catalyst 10: Mo₁₂Bi_{0.7}Co_{5.5}Fe_{1.5}K_{0.05}

In the catalyst 10, the crystallinity T was 5.9% and the crystallinity ratio R was 0.14.

The obtained catalyst 10 was filled in the same manner as in Experiment Example 1 and oxidation reaction of propylene was conducted under the same conditions as in Experiment Example 1. The results are shown in Table 1. In Experiment Example 10, the initial yield was a relatively high value of 92.2 mol %.

(11) Experiment Example 11

A catalyst 11 was prepared in the same manner as in Experiment Example 6, except that the amounts of bismuth nitrate, cobalt nitrate, iron nitrate and nickel nitrate were changed to 229 parts, 412 parts, 172 parts and 137 parts, and the periods for adding the solutions B-1 and B-2 to the solution A were respectively changed to 30 seconds in Experiment Example 6. The supported ratio of the catalyst 11 was 104 mass %, and the catalyst 11 had the following metal element composition excluding oxygen.

### Catalyst 11: Mo₁₂Bi₂Co₆Fe_{1.8}Ni₂K_{0.07}

In the catalyst 11, the crystallinity T was 16.2% and the crystallinity ratio R was 0.03.

The obtained catalyst 11 was filled in the same manner as in Experiment Example 1 and oxidation reaction of propylene was conducted under the same conditions as in Experiment Example 1. The results are shown in Table 1. In Experiment Example 11, the initial yield was a relatively high value of 92.2 mol %.

TABLE 1

| | | | | Passage | Reaction | Conversion Rate | Yield of |
|---|---|---|---|---|---|---|---|
| | Catalyst | Crystallinity | Crystallinity | of Time | Temperature | of Propylene | Acrylic Acid + |
| | No. | T (%) | Ratio R | (hours) | (° C.) | (mol %) | Acrolein (mol %) |
| Experiment | 1 | 7.1 | 0.10 | initial | 310 | 98.4 | 92.8 |
| Example 1 | | | | 4000 | 314 | 98.4 | 92.6 |
| Experiment | 2 | 18.9 | 0.02 | initial | 310 | 97.5 | 90.7 |
| Example 2 | | | | 4000 | 326 | 97.3 | 89.2 |
| Experiment | 3 | 14.3 | 0.06 | initial | 310 | 98.5 | 93.0 |
| Example 3 | | | | | | | |
| Experiment | 4 | 4.5 | 0.21 | initial | 310 | 97.8 | 91.7 |
| Example 4 | | | | 4000 | 317 | 97.7 | 90.9 |
| Experiment | 5 | 3.4 | 0.22 | initial | 310 | 96.6 | 89.6 |
| Example 5 | | | | 4000 | 320 | 96.4 | 87.5 |
| Experiment | 6 | 17.1 | 0.02 | initial | 310 | 97.7 | 91.4 |
| Example 6 | | | | | | | |
| Experiment | 7 | 3.4 | 0.01 | initial | 310 | 97.1 | 90.4 |
| Example 7 | | | | | | | |
| Experiment | 8 | 9.7 | 0.04 | initial | 310 | 98.0 | 92.6 |
| Example 8 | | | | | | | |
| Experiment | 9 | 7.9 | 0.12 | initial | 310 | 98.4 | 93.0 |
| Example 9 | | | | | | | |
| Experiment | 10 | 5.9 | 0.14 | initial | 310 | 98.1 | 92.2 |
| Example 10 | | | | | | | |
| Experiment | 11 | 16.2 | 0.03 | initial | 310 | 98.2 | 92.2 |
| Example 11 | | | | | | | |

(12) Experiment Example 12

(12-1) Preparation of Catalyst

A catalyst 12 was prepared in the same manner as in Experiment Example 1, except that the carrier used in Experiment Example 1 was changed to a silica-alumina spherical carrier having an average particle diameter of 6.5 mm. The supported ratio of the catalyst 12 was 100 mass %, and the metal element composition, the crystallinity T and the crystallinity ratio R of the catalyst 12 were the same as those of the catalyst 1.

(12-2) Reaction

A reactor having a reaction tube (reaction tube length: 3,000 mm, inner diameter: 25 mm) made of stainless steel and a shell in which a heat medium is flowed and which covers the reaction tube was vertically-installed. The catalysts 12 and 3 were filled into the reactor by sequentially dropping the catalyst 12 and the catalyst 3 from the top of the reaction tube so that the layer lengths come to be 800 mm for the catalyst 12 and 2100 mm for the catalyst 3.

While maintaining the temperature of the heat medium (a reaction temperature) at 315° C., a mixed gas consisting of 8 volume % of propylene, 15 volume % of oxygen, 8 volume % of steam and an inert gas such as nitrogen as the residue was introduced from the bottom of the reaction tube filled with the catalysts at a space velocity (STP) of 1950 hr⁻¹, thereby conducting oxidation reaction of propylene. In Experiment Example 12, the catalyst 12 was placed on an inlet side, with respect to the flow of the mixed gas, of the reaction tube, and the catalyst 3 was placed on an outlet side, with respect to the flow of the mixed gas, of the reaction tube. The reaction was conducted continuously for 4000 hours while adjusting the reaction temperature appropriately. The results are shown in Table 2. In Experiment Example 12, two kinds of catalysts, i.e. the catalysts 3 and 12, each of which has the crystallinity ratio R in the range of 0.03 or more and 0.20 or less, were filled into the reaction tube. The catalyst 12 having the higher crystallinity ratio R was placed on the inlet side, and the catalyst 3 having the lower crystallinity ratio R was placed on the outlet side. As a result, the yield was a high value of 92.5 mol % or more both in the initial stage and after 4000 hours.

(13) Experiment Example 13

A catalyst 13 was prepared in the same manner as in Experiment Example 3, except that the carrier used in Experiment Example 3 was changed to a silica-alumina spherical carrier having an average particle diameter of 6.5 mm. The supported ratio of the catalyst 13 was 100 mass %, and the metal element composition, the crystallinity T and the crystallinity ratio R of the catalyst 13 were the same as those of the catalyst 3.

Into the same reaction tube as in the Experiment Example 12, the catalysts 13 and 1 were filled by sequentially dropping the catalyst 13 and the catalyst 1 from the top of the reaction tube so that the layer lengths come to be 800 mm for the catalyst 13 and 2100 mm for the catalyst 1, and oxidation reaction of propylene was conducted under the same conditions as in Experiment Example 12. In Experiment Example 13, the catalyst 13 was placed on the inlet side, with respect to the flow of the mixed gas, of the reaction tube, and the catalyst 1 was placed on the outlet side, with respect to the flow of the mixed gas, of the reaction tube. The results are shown in Table 2. In Experiment Example 13, two kinds of catalysts, i.e. the catalysts 1 and 13, each of which has the crystallinity ratio R in the range of 0.03 or more and 0.20 or less, were filled into the reaction tube. The catalyst 13 having the lower crystallinity ratio R was placed on the inlet side, and the catalyst 1 having the higher crystallinity ratio R was placed on the outlet side. Also in Experiment Example 13, the yield was maintained to be high both in the initial stage and after 4000 hours; however, the performance in Experiment Example 13 was lower than that in Experiment Example 12.

(14) Experiment Example 14

A catalyst 14 was prepared in the same manner as in Experiment Example 2, except that the carrier used in Experiment Example 2 was changed to a silica-alumina spherical carrier having an average particle diameter of 6.5 mm. The supported ratio of the catalyst 14 was 100 mass %, and the metal element composition, the crystallinity T and the crystallinity ratio R of the catalyst 14 were the same as those of the catalyst 2.

Into the same reaction tube as in the Experiment Example 12, the catalysts 14 and 2 were filled by sequentially dropping the catalyst 14 and the catalyst 2 from the top of the reaction tube so that the layer lengths come to be 800 mm for the catalyst 14 and 2100 mm for the catalyst 2, and oxidation reaction of propylene was conducted under the same conditions as in Experiment Example 12. In Experiment Example 14, the catalyst 14 was placed on the inlet side, with respect to the flow of the mixed gas, of the reaction tube, and the catalyst 2 was placed on the outlet side, with respect to the flow of the mixed gas, of the reaction tube. The results are shown in Table 2. In Experiment Example 14, the catalysts 2 and 14, which have the same crystallinity ratio R each other out of the range of 0.03 to 0.20, were filled into the reaction tube. The yield in Experiment Example 14 was lower than those in the Experiment Examples 12 and 13.

(15) Experiment Example 15

A catalyst 15 was prepared in the same manner as in Experiment Example 5, except that the carrier used in Experiment Example 5 was changed to a silica-alumina spherical carrier having an average particle diameter of 6.5 mm. The supported ratio of the catalyst 15 was 100 mass %, and the metal element composition, the crystallinity T and the crystallinity ratio R of the catalyst 15 were the same as those of the catalyst 5.

Into the same reaction tube as in the Experiment Example 12, the catalysts 15 and 2 were filled by sequentially dropping the catalyst 15 and the catalyst 2 from the top of the reaction tube so that the layer lengths come to be 800 mm for the catalyst 15 and 2100 mm for the catalyst 2, and oxidation reaction of propylene was conducted under the same conditions as in Experiment Example 12. In Experiment Example 15, the catalyst 15 was placed on the inlet side, with respect to the flow of the mixed gas, of the reaction tube, and the catalyst 2 was placed on the outlet side, with respect to the flow of the mixed gas, of the reaction tube. The results are shown in Table 2. In Experiment Example 15, two kinds of catalysts, i.e. the catalysts 2 and 15, each of which has the crystallinity ratio R out of the range of 0.03 to 0.20, were filled into the reaction tube. The catalyst 15 having the higher crystallinity ratio R was placed on the inlet side, and the catalyst 2 having the lower crystallinity ratio R was placed on the outlet side. The yield in Experiment Example 15 was lower than those in the Experiment Examples 12 and 13.

TABLE 2

| | | Passage | Reaction | Conversion Rate | Yield of |
|---|---|---|---|---|---|
| | | of Time | Temperature | of Propylene | Acrylic Acid + |
| | | (hours) | (° C.) | (mol %) | Acrolein (mol %) |
| Experiment | inlet side: catalyst 12 (R = 0.10) | initial | 315 | 98.3 | 92.8 |
| Example 12 | outlet side: catalyst 3 (R = 0.06) | 4000 | 320 | 98.3 | 92.5 |
| Experiment | inlet side: catalyst 13 (R = 0.06) | initial | 315 | 98.1 | 92.4 |
| Example 13 | outlet side: catalyst 1 (R = 0.10) | 4000 | 321 | 98.0 | 92.1 |
| Experiment | inlet side: catalyst 14 (R = 0.02) | initial | 315 | 97.2 | 89.6 |
| Example 14 | outlet side: catalyst 2 (R = 0.02) | 4000 | 335 | 96.9 | 88.1 |
| Experiment | inlet side: catalyst 15 (R = 0.22) | initial | 315 | 97.4 | 89.8 |
| Example 15 | outlet side: catalyst 2 (R = 0.02) | 4000 | 334 | 97.2 | 88.2 |

1. A catalyst for producing acrolein and/or acrylic acid from propylene by gas-phase catalytic oxidation with molecular oxygen-containing gas, comprising
a catalytic active component containing molybdenum, bismuth and cobalt as essential elements, and having a crystallinity T of 4% or more and 18% or less in the 2θ range of 5° to 90°, measured by an X-ray diffraction analysis with Cu—Kα radiation.

2. The catalyst for producing acrolein and/or acrylic acid according to claim 1, wherein a crystallinity ratio R expressed by the following formula (1) is in the range of 0.03 or more and 0.20 or less,
R=M/T (1)
(wherein M represents a crystallinity of the peak at 2θ=26.5±0.5° in the catalytic active component, measured by the X-ray diffraction analysis with Cu—Kα radiation.)radiation.

3. The catalyst for producing acrolein and/or acrylic acid according to claim 1, further comprising an inert carrier for supporting the catalytic active component.

4. A process for producing acrolein and/or acrylic acid by gas-phase catalytic oxidation of propylene with molecular oxygen, comprising the step of conducting the gas-phase catalytic oxidation in the presence of the catalyst according to claim 1.

5. The process for producing acrolein and/or acrylic acid according to claim 4, wherein the catalyst is filled in a reaction tube of a fixed-bed reactor.

6. The process for producing acrolein and/or acrylic acid according to claim 5, wherein
the reaction tube has a plurality of reaction zones divided in an axis direction of the reaction tube, and
the respective reaction zones are filled with the catalysts having the different crystallinity ratio R from each other.

7. The process for producing acrolein and/or acrylic acid according to claim 6, wherein
the crystallinity ratios R of the plurality of the reaction zones decreases in an order from an inlet to an outlet of a raw material gas containing propylene and molecular oxygen-containing gas.
